(19) United States
(12) Patent Application Publication
(10) Pub. No.: US 2016/0310033 A1
(43) Pub. Date: Oct. 27, 2016

(54) EMG ASSISTANT: A method for the automated localization of root / plexus and/or other focal nerve damage in the upper and the lower extremities using either the routine clinical-neurological or the electromyographic muscle examination

(71) Applicants: Israel Yaar, Sharon, MA (US); Abraham Michael Yaar, Sharon, MA (US)

(72) Inventors: Israel Yaar, Sharon, MA (US); Abraham Michael Yaar, Sharon, MA (US)

(21) Appl. No.: 14/693,968

(22) Filed: Apr. 23, 2015

(51) Int. Cl. A61B 5/0488

(52) U.S. Cl. CPC A61B 5/0488

(57) ABSTRACT

A novel computerized method for automatic diagnosis of clinical-neurological and/or electromyographic (needle EMG) studies is presented. The clinician—neurologist, physiatrist, physical therapist or qualified other—performs a routine clinical and/or electromyographic examination of the patient's muscles and assigns graded levels of abnormality to each one of the muscles examined. This data, usually numbers in the range of 0 to 3, is input into the program. Based on the muscles examined and their abnormality levels the program finds the minimal location(s) of nerve-damage that explains the muscle findings, i.e. a diagnosis. Several approaches and techniques that were developed and utilized in the program are described below. Also, the program will compute and suggest to the clinician the additional name(s) of the next-best-muscle(s) to study in case he/she wants to improve the study results.

### CROSS-REFERENCE TO RELATED APPLICATION

None.

### STATEMENT REGARDING FEDERALLY SPONSORED RESEARCH OR DEVELOPMENT

Not applicable.

### REFERENCE TO SEQUENCE LISTING

Not applicable.

### DESCRIPTION OF THE SEVERAL VIEWS OF THE DRAWINGS

### Abbreviations

### Upper Extremity Muscles

BR Brachioradialis

ECR Extensor Carpiradialis

PT Pronator Teres

FCU Flexor Carpi Ulnaris

FDP Ulnar Flexor Digitorum Profundus

FCR Flexor Carpi Radialis

FDS Flexor Digitorum Sublimis

FDP Median Flexor Digitorum Profundus, Median innervated

FPL Flexor Pollicis Longus

PQ Pronator Quadratus

ECU Extensor Carpi Ulnaris

EDC Extensor Digitorum Communis

EIP Extensor Indicis Proprius

ADM Abductor Digiti Minimi

FDI First Dorsal Interosseous

APB Abdductor Pollicis Brevis

### Upper Extremity Nerve-Segments

Median@Wrist Median Nerve at the Wrist

Ulnar@Wrist Ulnar Nerve at the Wrist

Radial@Supinator Radial Nerve at the Supinator Muscle

Ant. Interosseous Anterior Interosseus Nerve

Median@Pronator Median Nerve at the Pronator Muscle

Ulnar@FCU Ulnar Nerve at the Flexor Carpi Ulnaris Muscle

Ulnar@Above Elbow Unlar Nerve above the Elbow

Median@Low Arm Median Nerve in the lower Arm

Radian@Low Arm Radial Nerve in the lower Arm

Radial@Upper Arm Radial Nerve in the upper Arm

Musc. Cutaneous Musculocutaneous Nerve

### Lower Extremity Muscles

TFL Tensor Facie Latae

TA Tibialis Anterior

PL Peroneus Longus

PB Peroneus Brevis

FDL Flexor Digitorum Longus

FHL Flexor Hallucis Longus

EDL Extensor Digitorum Longus

EHL Extensor Hallucis Longus

EDB Extensor Digitorum Brevis

AH Abductor Hallucis

ADM Abductor Digiti Minimi

### Lower Extremity Nerve-Segments

SPxVD/Tibial Above knee Sacral Plexus Ventral Division & Tibial Nerve above knee

SPxDD Sacral Plexus Dorsal Division

LPxVD/Obturator Lumbar Plexus Ventral Division & Obturator Nerve

LPxDD/Femoral Lumbar Plexus Dorsal Division & Femoral Nerve

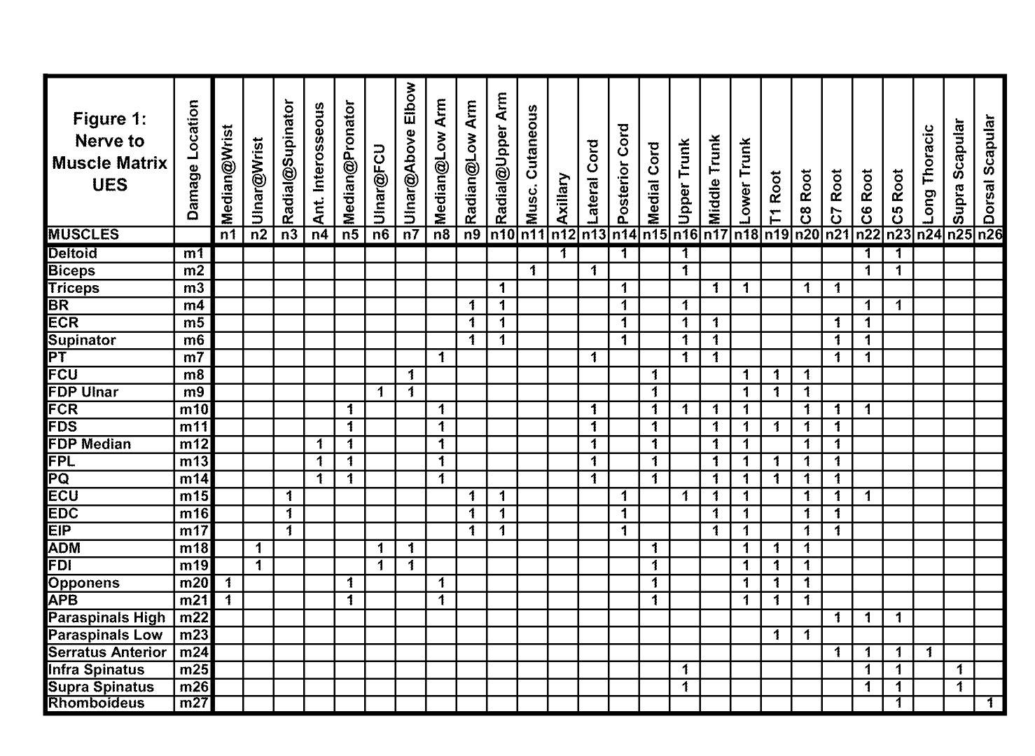

FIG. 1: Nerve-to-Muscle connections-matrix for the upper extremities (UES): The x-axis (top row) lists the abbreviated names of 26 nerve-locations (segments) within the upper-extremities nervous-system. These locations are assigned the names n1 to n26 (second row). For example, n1 is assigned to the segment of the median nerve at the wrist (Median@Wrist in the figure), and n26 is assigned to the dorsal scapular nerve.

The y-axis (leftmost column) lists the abbreviated names of the 27 muscles that are usually examined in the upper-extremities both clinically and/or electromyographically. These locations are assigned the names m1 to m27 (second column). For example, m1 is assigned to the Deltoid muscle, and m27 is assigned to the Rhomboideus muscle.

In this figure, the digit 1 signifies a nerve-to-muscle connection and is inserted in those cells where a nerve-segment on the x-axis innervates a muscle on the y-axis. Cells where the nerves in their x-axis do not innervate the related muscles on their y-axis are left empty. It is apparent that when a nerve is damaged the appropriate muscle will show clinical and electromyographically abnormalities and the appropriate cell will be marked with the digit 1. Therefore, the digit 1 also stands for damaged nerve-muscle pairs. As the table does not include a severity level for each damaged nerve, the value of 1 is a virtual value indicating only a state of abnormality. Cells of muscles that are not connected to injured nerves are left empty and later referred to with the value of 0.

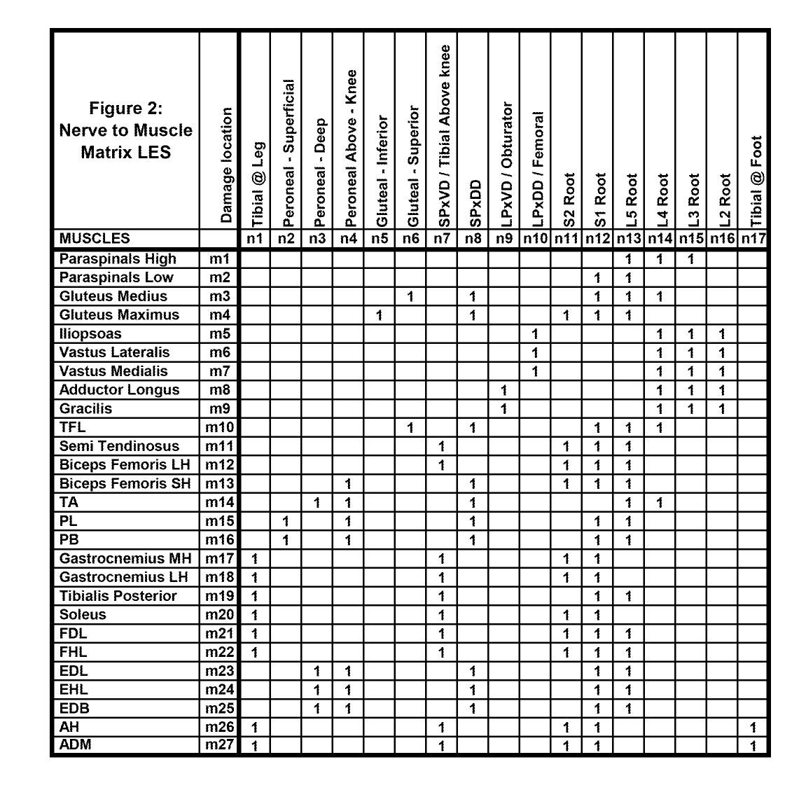

FIG. 2: Nerve to Muscle Matrix for the lower extremities (LES): Same rules as in FIG. 1, only that the lower-extremity matrix is composed of 17 nerves (n1 to n17) and 27 muscles (m1 to m27)

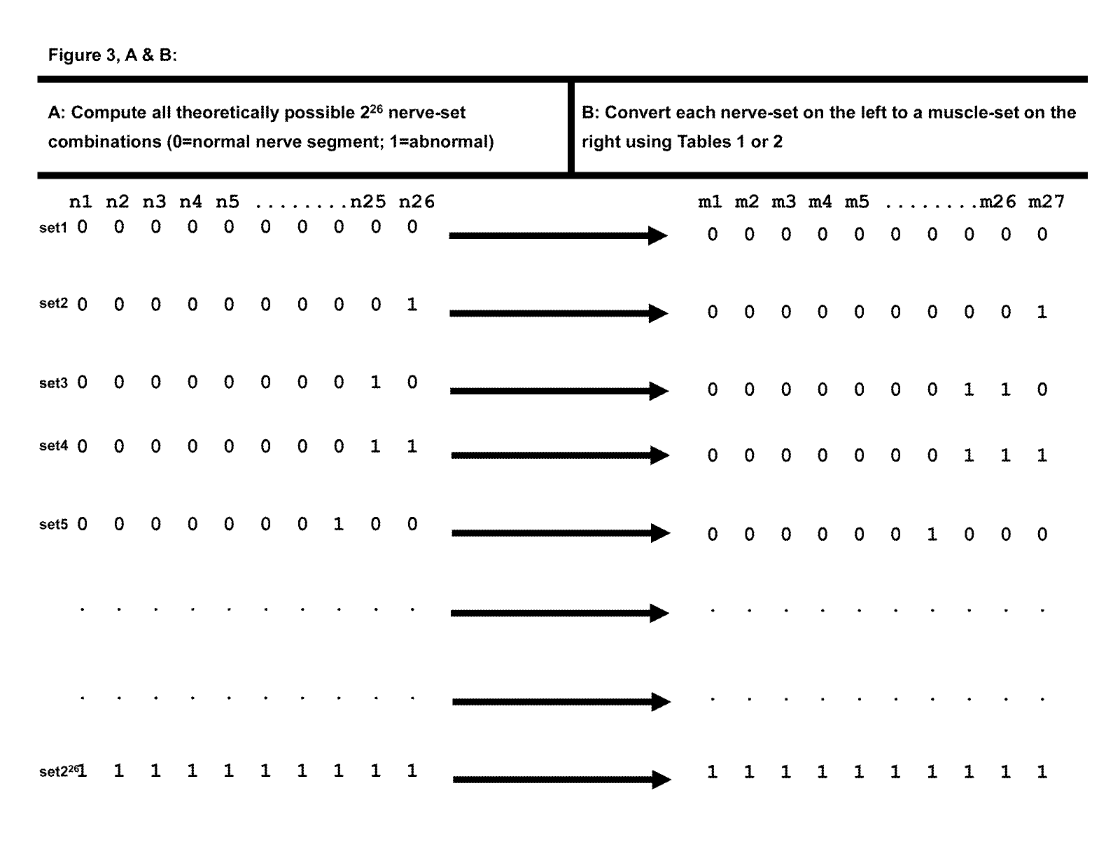

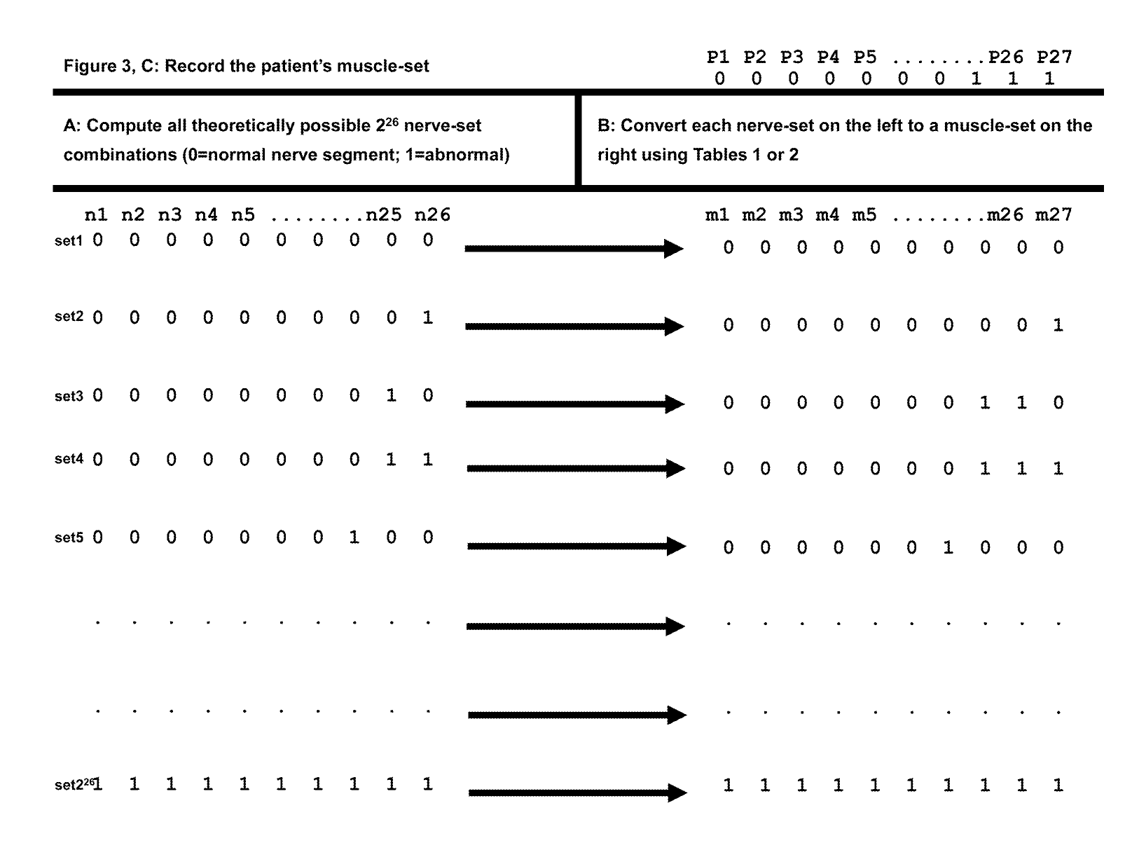

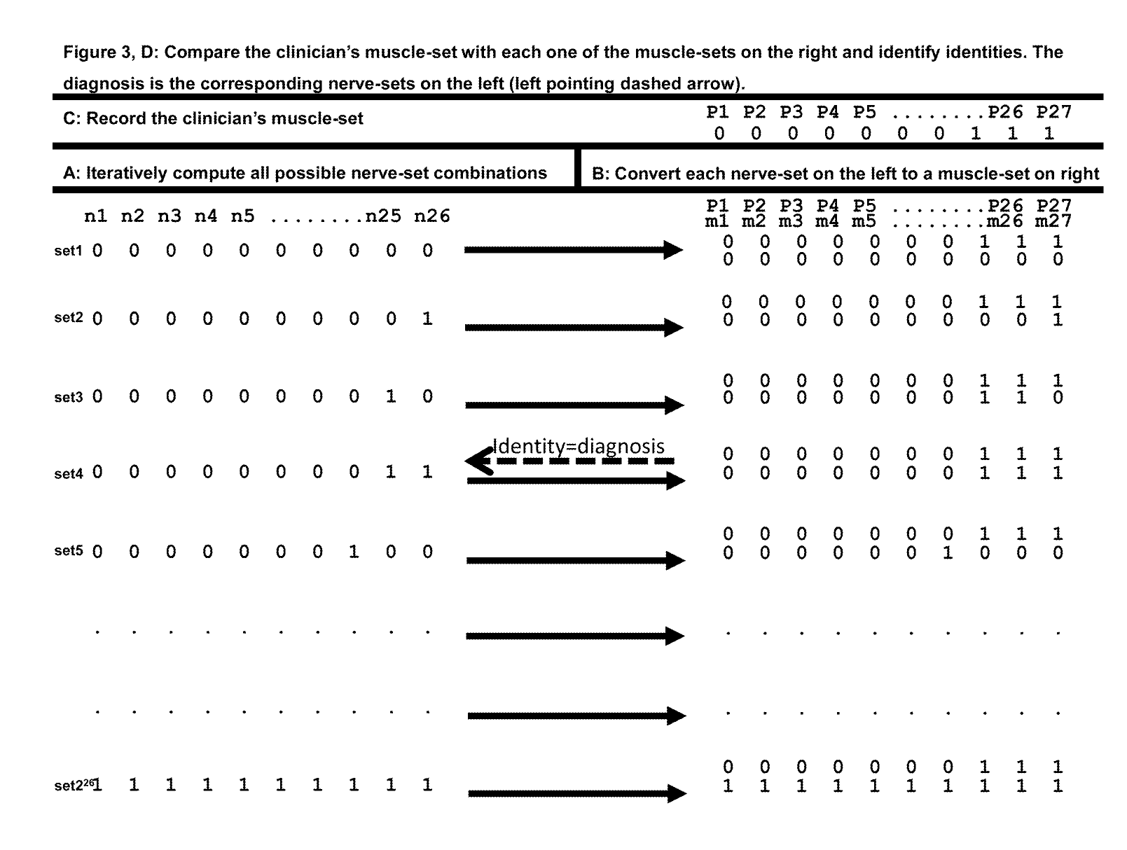

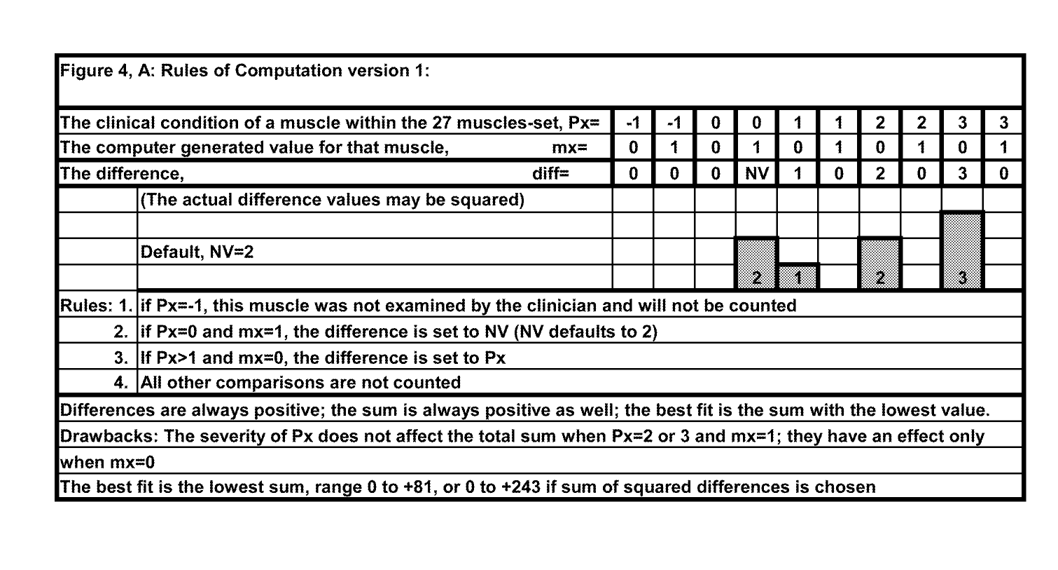

FIG. 3, A & B: Similar notations to those in FIGS. 1 & 2 are used; virtual nerve-sets are converted to virtual muscle-sets using FIG. 1 matrix. Here, the digit 0 indicates normality and the digit 1 indicates abnormality, nerve and muscle respectively.

FIG. 3, C: Similar notations to previous figures are used with the addition of P1 to P27 that stand for the clinician's empirical findings. For example, P1 represents damaged Deltoid while P27 represents damage Rhomboideus. These notations correlate with the notations of the virtual muscle-sets, m1 to m27.

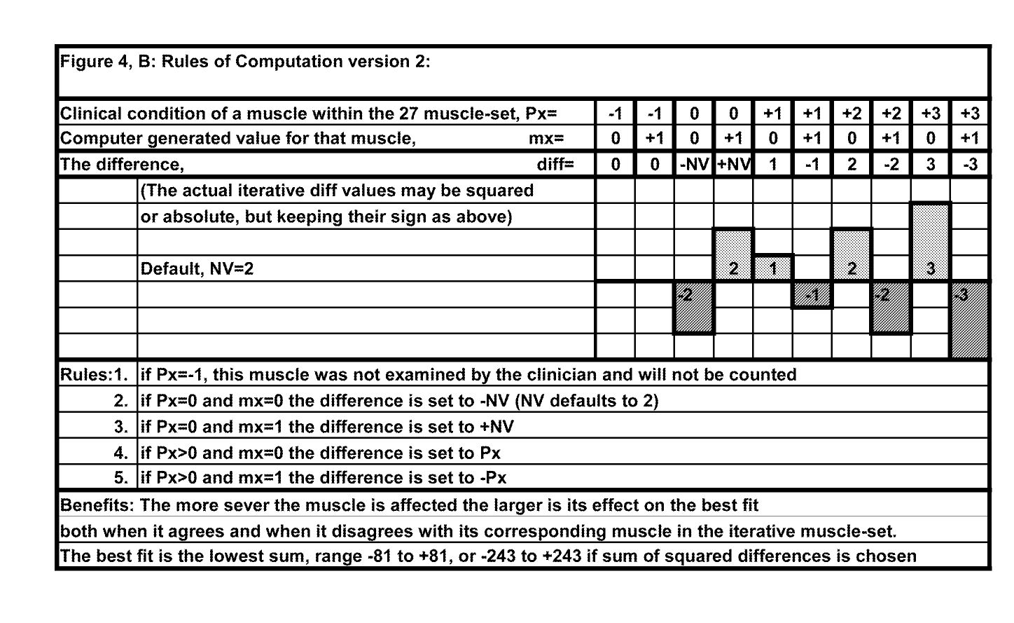

FIG. 3, D: The clinician's empirically derived muscle-set, P1 to P27, is iteratively compared to each and every one of the virtual muscle-sets, m1 to m27. The comparison technique is explained later in the text and in FIGS. 4A & B.

FIG. 4, A: This is the first version of the rules for computing sum-of-differences or sum-of-difference squared between the clinician's empirical muscle-set (P1 to P27) and each of the 2²⁶ virtual muscle-sets. These comparisons are made between the values of corresponding muscles, such as P1 with m1, P2 with m2 etc and these differences are added to a sum-total difference between each of the sets. The notations are similar to those used in the other figures with the addition of −1 which was assigned to muscles that were not examined by the clinician. Also, Px values 1, 2 and 3 are the empirical values of the severity of muscle-abnormalities that were found and such assigned by the clinician. A value of 1 indicating low certainty that the muscle is damaged, and a value of 3 indicating maximal certainty that the muscle is damaged, NV (normal value) is the strength the clinician assigns to her/his assertion that the muscle examined is normal; it defaults to the value 2.

FIG. 4, B: This is the second version of the rules for computing sum-of-difference or sum-of-difference squared; otherwise the notation are identical to those in FIG. 4, A.

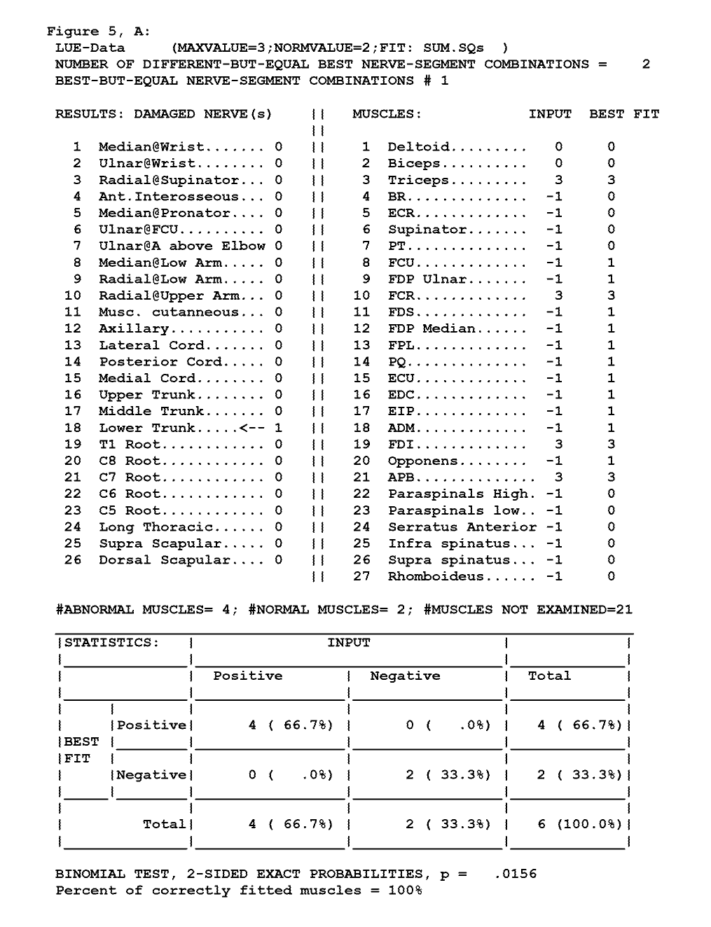

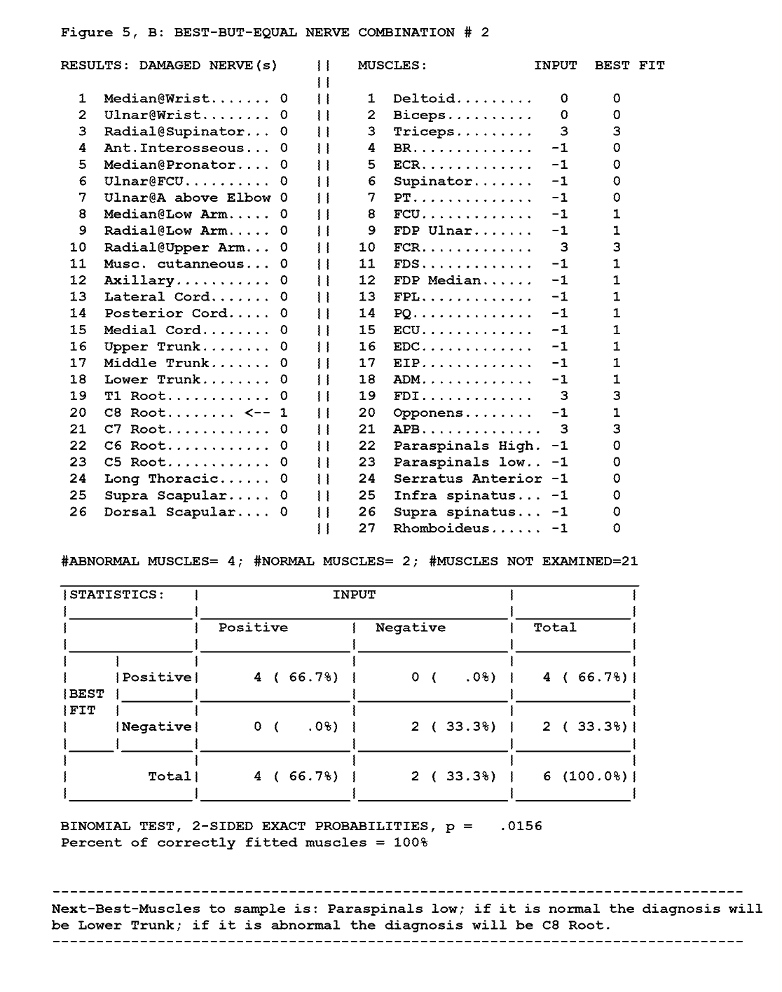

FIG. 5, A and FIG. 5, B: These figures show one way, but not the only way of presenting the EMG ASSISTANT's results. In this particular case, the clinician's empirical findings resulted in two possible diagnoses (combinations) that are of statistically identical strength, in FIG. 5, A it is the upper-trunk, while in FIG. 5, B it is C8 root that is damaged. The EMG ASSISTANT found that the Next-Best-Muscle to empirically sample should be Paraspi.low as is shown at the bottom paragraph of FIG. 5, B. And, of course, if Paraspi.low is found normal the one and only possible diagnosis is damage at the Lower Trunk, while if Paraspi.low is found abnormal the only possible diagnosis is damage to C8 root.

### BRIEF SUMMARY OF THE INVENTION

A novel computerized method for automatic diagnosis of clinical-neurological and/or electromyographic (needle EMG) studies is presented. The clinician—neurologist, physiatrist, physical therapist or qualified other—performs a routine clinical and/or electromyographic examination of the patient's muscles and assigns graded levels of abnormality to each one of the muscles examined. This data, usually numbers in the range of 0 to 3, is input into the program. Based on the muscles examined and their abnormality levels the program finds the minimal location(s) of nerve-damage that explains the muscle findings, i.e. a diagnosis. Several approaches and techniques that were developed and utilized in the program are described below. Also, the program will compute and suggest to the clinician the additional name(s) of the next-best-muscle(s) to study in case he/she wants to improve the study results.

### FIELD OF THE INVENTION

This invention relates to medical diagnosis of neuromuscular diseases specifically segmental damage to nerves that result in muscle weakness and other muscle abnormalities. The field overlaps both clinical neurological examination derived diagnosis and electromyographic diagnosis, either separate or combined. It also relates to automation of medical diagnosis. The clinical diagnostic work in this field is based on the known anatomical connections between nerves and muscles, referenced below.

### DESCRIPTION OF RELATED ART

Neurological clinical examination of the skeletal muscles includes the examination and recording of the strength of said muscles and whether or not there are signs of muscle wasting. Thereafter, the clinician grades her/his findings on a scale, usually from 0, for normal muscle to 3, for severely abnormal muscle. Thereafter, the clinician applies her/his learned knowledge and deduces from said muscle findings on what focal nerve damage could explain them, i.e. the diagnosis.

Needle-electromyography is an examination of said muscles by the recording of the muscle-fibers electrical activities using needle-electrodes. The electromyographer records these electrical activities and grades them for abnormality, usually on a scale of 0, for normal activities, to 3, for severely abnormal activities. Thereafter, the electromyographer applies her/his learned knowledge and deduces from said muscle findings on what focal nerve damage could explain her/his findings, i.e. the diagnosis.

The said neurological clinical-examination may be done and interpreted by any clinician—neurologists, physiatrists, physical therapists or any qualified others—however, the needle-electromyographic study is supposed to be done by a clinician qualified in the field of electromyography who usually perform the relevant clinical neurological-examination as well.

In either case, said deducing process that starts after assigning said levels of abnormalities to each of the muscle examined is extremely subjective, it depends on the individual capabilities of the interpreter, relies heavily on the individual's capability to memorize complicated nerves-to-muscles wiring schematics, the individual's deducing capabilities and the time allocated for the task. This is especially true when the nerve damage involves more than one nerve and/or when there are extraneous abnormalities in the study that may blur the otherwise expected clear patterns; it may confuse even the best of diagnosticians and make their diagnosis be fraught with errors. Also, and of genuine importance: Usually, the interpreter does not have another specialist with whom to discuss the case and get a second opinion. Automation of the diagnostic process may be helpful on all counts; it does not depend on the individual capabilities, it is fast, and it will provide an easily available and objective second opinion.

This patent proposal supersedes my (Israel Yaar's) U.S. Pat. No. 6,366,806. As will be shown below, the new patent proposal adds a computation mode that was not proposed before, it also adds a new computation shortcut that accelerates the computational speed by many folds and as such enables a computational intensive novel-initiative to find the next-best-muscle for the clinician to test in order to improve her/his results. This shortcut will also enable the analysis of much larger nerve/muscle matrices.

### REFERENCES

The Peripheral Nervous System Muscle Innervations Schematics have been known for many years; FIGS. 1 & 2 above were adapted from the following sources:
1. Medical Research Council, Memorandum No. 45: Aids to the examination of the peripheral nervous system, Crown, London, 1976.
2. E F Delagi and A O Perotto: Anatomic Guide for the Electromyographer, C C Thomas, Springfield, Ill. 1980.
3. R K Sethi and L L Thompson: The Electromyographer's Handbook, Little, Brown and company, Boston, 1989.
4. J Kimura: Electrodiagnosis in Diseases of Nerve and Muscle, F A Davis, Philadelphia, 1989.
5. J A Liveson: Peripheral Neurology, FA Davis Company, Philadelphia, 1991.
6. R D Adams and M Victor: Principles of Neurology, McGraw-Hill, New York, 1993.
7. A O Perotto: Anatomic Guide for the Electromyographer: The Limbs and Trunk, C C Thomas, Springfield, Ill., 1994.
8. L P Rowland: Merritt's Textbook of Neurology, Williams and Wilkins, Baltimore, 1995.
9. R J Joynt and R C Griggs: Clinical Neurology, Lippincott Williams and Wilkins, Philadelphia, 1998.

### DETAILED DESCRIPTION

The diagnosis of focal nerve damage is based in part on the evaluation of muscles, usually those that are clinically related to the patient's complaints. This examination can be a physical examination and/or electromyographic examination of said muscles. This examination will generate and record abnormality levels for each one of the muscles examined, usually in the range of 0 to 3, where zero stands for normal muscle and 3 stands for maximally damaged muscle. This range can be expanded.

Note: By design the EMG ASSISTANT interprets muscle data that was generated by the clinician; it does not interpret raw EMG signals. Also, it is designed to localize focal nerve damage and it is not diagnostic in polyneuropathy and not in myopathy.

The EMG ASSISTANT analysis of the patient's clinical findings is based on FIGS. 1 & 2. These Figures represent nerve-to-muscle connections in matrix formats and are the condensation of known anatomical and pathological data (references 1-9).

In the present version, the upper extremity (UES) data is condensed and limited to 27 muscles to be examined and 26 possible locations where upper extremity nerves may be damaged (Table 1). This system can be extended and applied to much bigger matrices; more muscles and more nerves. The locations that were chosen for the present version are those that are presently generally accepted nerve damage locations in clinical neurophysiology. Innervation-aberrations are not accounted for and have not been automated; they are left for the clinician to consider.

The lower extremity (LES) data is condensed and limited to 27 muscles that can be examined and 17 locations where lower extremity nerves may be damaged (Table 2). These locations are also the accepted nerve damage locations in clinical neurophysiology and can be extended to check more muscles and more nerves. Innervation-aberrations are not accounted for and have not been automated; they are left for the clinician to consider.

### Steps in the Process Relating to the Upper Extremities (UES):

Step 1 (FIG. 3, A): This step does not involve patient's data. The program begins by generating all the possible one-to-many virtually damaged nerve locations in the UES nerve-sets (1-to-26 possible locations, n1 to n26 in FIG. 1). This process will generate 2²⁶=67,108,864 nerve-sets. Each nerve set contains the names of 26 nerves—some of which are damaged, some of which are not. Each set is unique. Zero stands for a nerve location that is unaffected, i.e. normal, and One stands for a damaged nerve location within a set. As this in not empirical data and there is no information on the severity of the damage, it is restricted to 0s and 1s.

Step 2 (FIG. 3, B): This step does not involve patient's data. Each one of the 2²⁶ unique nerve-sets above is translated by the program using FIG. 1 into the theoretically expected muscle-sets (m1 to m27 in FIG. 1). This process will generate 2²⁶=67,108,864 sets of muscles. Within each set some of the muscles are normal, and some of the muscles are damaged. These 2²⁶ muscle-sets may not be unique as several combinations of nerve-sets may theoretically bring about the same muscle-set. Zero stands for muscles that are not affected by damaged nerves in the corresponding nerve-sets, i.e. normal, and One stands for abnormal muscles within the 27 muscles of each set. As this in not empirical data, it is restricted to 0s and 1s.

Step 3 (FIG. 3, C): The clinician examines the patient and records the actual muscle-set, P1-to-P27 (right upper line in the Figure), specific to this patient, and inputs it into the computer using the input dialog. At this stage in the explanation of the EMG ASSISTANT we assume that the clinician grades the patient's muscles in a restricted range of only 0s and 1s; see below for an expanded explanation.

Step 4 (FIG. 3, D): The program compares the actual patient's muscle-set (P1-to-P27) to everyone of the previously generated 2²⁶ muscle-sets (m1-to-m27). When identity is found—there might be more than one identity—the nerve-set that generated this identity is found as marked by the broken arrow (see more below): This is one of the possible diagnoses. Then, from all the possible diagnoses the program selects the nerve-set(s) with the smallest number of damaged nerves and reports it (them) as the final diagnosis.

The above stated Steps 3 & 4 are shown in order to simplify the reader's learning process. However, almost always the clinician will grade the muscles in a wide range of abnormalities, such a 0, 1, 2 and 3, and sometimes even into wider ranges. Therefore, the comparisons as done in FIG. 3, D do not amount to identities, since the comparisons of 0s and 1s to 2s and 3s cannot amount to identities. A diagnosis based on the clinical severity of the data is likely to be more reliable than that resulted from the restricted range of normal/abnormal (0s and 1s). The actual comparisons are based on either the sums of absolute-value differences or the sum-of-squared differences between sets of P1-to-P27 and m1-to-m27 following certain rules. The clinician can set the program to whichever rule he/she is comfortable with.

Step 5: See below under “OUTPUT.”

### Rules of Computation, Version 1 (FIG. 4, A)

Each one of the 2²⁶ computer generated virtual muscle-sets (m1-to-m27) is compared with the clinician's empirical muscle-set (P1-to-P27), one corresponding muscle at a time (m1 with P1 through m27 with P27, generalized to Px and mx), and for each pair (Px, mx) the 27 separate differences are added up to that set's sum-total:
a. If Px=−1, i.e. this muscle was not examined by the clinician and will not be counted.
b. If Px=0 and mx=1: The difference is set to NV (defaults to 2).
c. If Px>0 and mx=0: The difference if set to Px.
d. All other possible comparisons are not considered.
e. All comparisons above are added to give the sum-total for each of the computer's generated 2²⁶ muscle-sets.

### EXAMPLE

| | |
|---|---|
| Px: | −1 −1 0 0 1 1 2 2 3 3 −1 −1 0 0 1 1 2 2 3 3 −1 −1 0 0 1 1 2 |
| mx: | 0 1 0 1 0 1 0 1 0 1 0 1 0 1 0 1 0 1 0 1 0 1 0 1 0 1 0 |
| Diff: | 0 0 0 2 1 0 2 0 3 0 0 0 0 2 1 0 2 0 3 0 0 0 0 2 1 0 2 |

Sum − total = 21

f. This method's sum-total values can range from 27*3=81 (when each Px=3 and each mx=0) to 27*0=0 (when each Px>0 and each mx=1).
g. The muscle-sets with the smallest sum-totals are considered best fit and are converted back to the nerve-sets that could have generated them. There may be more than one such nerve-set).
h. From the latter, the one or the few nerve-sets with the smallest number of damaged nerves are output as the diagnoses.

The rules of comparison for sum of squared-differences are identical to the above only that the sum is the sum of the squared-differences. In the example above with squared-differences the sum-total=45.

The logic behind these rules is as follows: As mx can be either 0 or 1, where 1 indicates abnormal muscle and 0 indicates normal muscle, supposedly we cannot measure the difference between any empirical grade of abnormality higher than 1 and that of virtual 1; however the differences when Px>0 and mx=0 is measurable by the certainty of the examiner that that muscle is abnormal and its abnormality equals Px. Also, the difference when Px=0 and mx=1 is measurable and is the level of the clinician's confidence in his statement that the muscle (Px) is normal when compared to the computer generated value of 1. In this software that confidence is called NV (Normal Value) and defaults to 2.

### Rules of Computation, Version 2 (FIG. 4, B)

Each one of the computer generated 2²⁶ virtual muscle-sets is compared with the clinician's actual muscle-set, one corresponding muscle at a time (m1 with P1 through m27 with P27, generalized as Px and mx) and for each compared set-pair the 27 separate differences are added-up to that set's sum-total:
a. If Px=−1, this muscle was not examined and will not be counted.
b. If Px=0 and mx=0: The difference is set to −NV (NV defaults to 2).
c. If Px=0 and mx=1: The difference is set to +NV
d. If Px>0 and mx=0: The difference is set to Px.
e. If Px>0 and mx=1: The difference is set to −Pk.
f. All comparisons above are added to give a sum-total for each of the computer's generated 2²⁶ muscle-sets.
   When this set of rules is applied to the example above it will change as follows:

| | |
|---|---|
| Px: | −1 −1 0 0 1 1 2 2 3 3 −1 −1 0 0 1 1 2 2 3 3 −1 −1 0 0 1 1 2 |
| mx: | 0 1 0 1 0 1 0 1 0 1 0 1 0 1 0 1 0 1 0 1 0 1 0 1 0 1 0 |
| Diff: | 0 0 −2 2 1 −1 2 −2 3 −3 0 0 −2 2 1 −1 2 −2 3 −3 0 0 −2 2 1 −1 2 |

Sum − total = 2

g. This method's sum-total values can range from 27*3=81 (when each Px=3 and each mx=0) to 27*(−3)=−81 (when each Px=3 and each mx=1). The muscle-sets smallest sum-total are considered best fit and are converted back to the nerve-sets that could have generated them (note that there may be more than one such nerve-set).
h. From the latter, the one or the few nerve-sets with the smallest number of damaged nerves are output as the diagnoses.

The rules of comparison for sum-of-squared differences are identical to the above only that the sum-total is the sum of the squared-differences—however, each squared difference retains its original sign (!); in the example above the sum-total=4.

The logic behind this method is as follows: The aim is to build a system tuned to the severity of the abnormalities as stated by the clinician and to include those occurrences when Px>1 and mx=1 and those occurrences where Px=0 and mx=0. Those occurrences have no effect when version 1 of the rules of computation is applied (FIG. 4, A); the current rules will result in the inclusion of all the clinician data and levels of abnormalities. It is expected that these rules will enhance the success of the EMG ASSISTANT as a diagnostic system.

In order to implement said logic, agreement between Px and mx will generate negative values and will reduce the sum-total for each said comparison above, while a disagreement will generate a positive value that will increase the sum-total of each said comparisons above, and the best fit between the clinician's data and the computer generated virtual muscle-sets is the smallest sum.

Steps in the process relating to the lower extremities (LES):

These steps are identical to those of the UES in the confines of FIG. 2: 27 muscles and 17 possible focal nerve-damage locations, amounting to only 2¹⁷ (131,072) unique nerve-set combinations. Otherwise, the approach and computations are identical and will not be repeated here.

### Additional Techniques

There is one variation to the above mentioned technique of generating and analyzing a 26×27 UES matrix (26 nerves by 27 muscles) or the 17×27 LES matrix (17 nerves by 27 muscles. This new technique accelerates the computational speed of the EMG ASSISTANT by many folds and gives the same diagnostic results, and can even accelerate matrices that are even much larger than 26×27, if more nerves and more muscles need be studied. First brief rehashing of the original technique as presented above in FIGS. 3, A to D: The program actually generates all the 2²⁶ virtual and unique nerve-sets and converts each one to muscle-sets which are then compared one muscle at a time with the recorded clinician's muscle-set, the best-fits are converted back into nerve-set(s), and the diagnosis is the nerve-set(s) with the smallest number of damaged nerves. All this is done on the fly in real time and is lengthy. However, even though there are 2²⁶ unique nerve-sets, there is not necessarily the same number of unique muscle-sets as the anatomical wirings of nerves-to-muscles force a smaller number of unique muscle-sets. This issue was studied and it was found that there can be only 5099 unique muscle-sets in the UES as presented in Table 1. The latter finding means that some muscle-sets can be generated by multiple combinations of unique nerve-sets.

Therefore, another computational path was designed to take advantage of the above, as follows: FIG. 3, A & B are computed as before only once, before the clinical study. Then the matrix in FIG. 3, B is searched for repetitions of the same muscle-set. When found, the nerve-set corresponding to it is kept in another matrix which is headed by that muscle-set. Thereafter, all repetitions are erased. We are left with 5099 unique muscle-sets and their varied number of unique nerve-sets that are possible diagnoses. As a consequence, the actual process of making a diagnosis, comparable to FIG. 3, D involve comparing—applying the various comparison-techniques above—only 5099 muscle-sets to the clinician's empirical set and when the best fit is found it is already coupled with the possible diagnoses. The last step is just like similar steps described before. Among those coupled diagnoses the one or several with the smallest number of damaged-nerves are chosen and output as the final diagnoses.

### Output

Once a diagnosis is made, i.e. nerve-set(s) comprised of 26 nerves or nerve-segment names, some are marked as normal and some as damaged is output and the following statistics are computed and output with it:
a. The damaged nerves are marked as in the example culminating in FIG. 5, A & B. There are two possible diagnostic nerve-sets (combination #1 and combination #2). In each, only one nerve was found damaged and marked with an arrow. In combination #1, it is the lower trunk; while in combination #2 it is the C8 Root.
b. Each diagnostic nerve-set is converted to a muscle-set using FIG. 1 or 2, respectively.
This muscle-set is called “Best-fit”. Ideally, it will be identical to the clinician's muscle-set, named “Input”. The closer they are the more reliable the diagnosis is.
c. In each of the combinations (#1 and #2), separately, these two muscle-sets are numerically compared to each other to establish how close to the ideal fit they are. These numerical comparisons are as follows:
d. A truth table summarizing the quality of said fit into four groups: True positives, false positives, true negatives and false negatives.
e. A 2-sided exact probability Binomial Test p-value is computed comparing the recorded muscle-set and the best fit muscle-set. This statistic takes into account the total number of muscles that were studied, the number of muscles correctly classified (true-positives and true-negatives), and the number of muscles incorrectly classified (false-positives and false-negatives). The p-value indicates the likelihood that the conversion muscle-set best fit in the table and the original recorded muscle-set relate to each other better than just by chance.
f. The percentage of correctly classified muscles is computed as 100*[(true-positives+true-negatives)/(total number of muscles examined)].

Step 5: Computing the next-best-muscle to sample.

The clinician may be unhappy with the results, such as when the resulting diagnosis is given an unsatisfactory p-value, or when the computations end with too many possible diagnoses (combinations) which is not very helpful whether statistically significant or not, or if for whatever reason the clinician wants to sample additional muscles. In such cases the EMG ASSISTANT can compute and advise the clinician which is the next-best-muscle to sample.

This computational decision is done as follows: The EMG ASSISTANT takes the muscle-set that was sampled by the clinician and adds to it one muscle from those muscles that were not sampled, once as if the added muscle is abnormal, for example +2, and once as if the added muscle is normal (0). Then it computes and keeps the usual statistics as in FIG. 5. Once the computations for the added muscle are completed, said muscle is dropped and a different muscle that previously was not recorded by the clinician is added, and the process repeats. At the end of this process the muscle that has the best statistics (see below) both when presented as abnormal and when presented as normal is the one suggested to the clinician as the next-best-muscle to sample. There may be more than one. There may be none, in which case the program reports in two columns, one column for the next-best-muscle or muscles that generate(s) the best statistics when abnormal and the other column for the next-best-muscle or muscles that generate(s) the best statistics when normal. The clinician is advised to take one muscle from each column to sample.

Said statistics include:
1. The binomial-test p-value, subsequently abbreviated as ‘p’,
2. The number of possible correct diagnoses, also named combinations in FIG. 5, subsequently be abbreviated as ‘nComb’, and
3. The percentage of correctly fitted muscles, subsequently abbreviated as ‘%’.

The actual decision making process goes as follows: Said muscles that are added and dropped are assumed abnormal, grade +2. For each one of said muscles the three statistics ‘p’, ‘nComb’ and ‘%’ are computed. These statistics are entered in one list.

Then, said muscles that are added and dropped are assumed to be normal, grade 0. For each one of said muscles the three statistics ‘p’, ‘nComb’ and ‘%’ are computed. These statistics are entered into another list
a. Thereafter, the program will seek those muscles for which ‘p’ & ‘nComb’ & ‘%’ have the best values in both lists and report them as the next-best-muscle (or muscles) to sample. If there are more than one the clinician will choose his preferred one.
b. If the criteria in (a) above could not be met, then the program will screen said muscles statistics for muscles with smallest ‘nComb’ and then, among the chosen ones, the program will select again those with the best ‘p’ & ‘%’ and report them as the next-best-muscle (or muscles) to sample.
c. If none of the muscles shows on both lists, the clinician is expected to choose one best muscle from each list and sample them both.

The next-best-muscle suggestion is reported in FIG. 5,B last paragraph.

What we claim as our invention is:

1. (canceled)

2. (canceled)

3. (canceled)

4. (canceled)

5. (canceled)

6. (canceled)

7. (canceled)

8. (canceled)

9. A method for the automated localization of nerve damage based on the rating of the patient's clinical and/or electromyographic muscle status.

10. The method of claim 9, wherein nerve-to-muscle connection schematics as presented in FIGS. 1 & 2 are utilized. Furthermore, the method of claim 9 applies to any variation of said figures' number and sequence of nerves and muscles.

11. The method of claim 9, wherein the process utilizes the generation of 2̂^{(the number of nerve locations)} unique nerve-sets and their translation into 2̂^{(the number of nerve locations)} non-unique muscle-sets as presented in FIG. 3, A and B for the upper extremities with similar process for the lower extremities.

12. The method of claim 9, wherein the patient's rated muscle-set is compared with each one of the 2̂^{(the number of nerve locations)} non-unique muscle-sets as presented in FIG. 3, D. Identities point to the correct diagnosis.

13. The method of claim 9, wherein the patient's rated muscle-set is compared with each one of the 2̂^{(the number of nerve locations)} non-unique muscle-sets as presented in FIG. 3, D. Identities are decided based on computation version 1 as presented in FIG. 4, A and point to the correct diagnosis. FIG. 4, A is an example of a rating system that extends from 0 to 3; however, the method of claim 9 applies to any rating scheme.

14. The method of claim 9, wherein the patient's rated muscle-set is compared with each one of the 2̂^{(the number of nerve locations)} non-unique muscle-sets as presented in FIG. 3, D. Identities are decided based on computation version 2 as presented in FIG. 4, B and point to the correct diagnosis. FIG. 4, B is an example of a rating system that extends from 0 to 3; however, the method of claim 9 applies to any rating scheme.

15. The method of claim 9, wherein the patient's rated muscle-set is compared with each one of the 2̂^{(the number of nerve locations)} non-unique muscle-sets as presented in FIG. 3, D. Identities are decided based on the computation of any linear and/or non-linear goodness of fit statistics.

16. The method of claim 9, wherein techniques for accelerating the computational speed are utilized.

17. The method of claim 9, wherein application of the techniques above to matrices larger than 26 nerves by 27 muscles for the upper extremities and 17 nerves by 27 muscles for the lower extremities is utilized.

18. The method of claim 9, wherein the report-format as presented in FIG. 5, A & B is given as example but not restricted to it.

19. The method of claim 9, wherein the statistical methods in validating the diagnosis as presented in FIG. 5, A & B are presented as examples but not restricted to them.

20. A method for finding the next-best-muscle for the clinician to sample.

21. The method of claim 20, wherein the program accepts into its process all the muscles that were sampled by the clinician with their grading and then adds one of the muscles that were not sampled to that group, once assumed normal and once assumed abnormal, and computes the now expanded group statistics. Then that added muscle is dropped and another muscle that was not sampled added as above and the process repeats. At the end of this process the clinician is prompted to sample that muscle (or muscles) that got the best statistics.
